(19) United States
(12) Patent Application Publication
(10) Pub. No.: US 2006/0099279 A1
(43) Pub. Date: May 11, 2006

(54) Novel anti-diabetic herbal formulation

(75) Inventors: Palpu Pushpangadan, Uttar Pradesh (IN); Chandana Venkateswara Rao, Uttar Pradesh (IN); Ajay Kumar Singh Rawat, Uttar Pradesh (IN); Dadala Vijay Kumar, Andhra Pradesh (IN)

Correspondence Address: LADAS & PARRY, 26 WEST 61ST STREET, NEW YORK, NY, 10023, US

(73) Assignee: COUNCIL OF SCIENTIFIC & INDUSTRIAL RESEARCH

(21) Appl. No.: 10/984,947

(22) Filed: Nov. 9, 2004

(51) Int. Cl. A61K 36/9068; A61K 36/48

(52) U.S. Cl. 424/756; 424/757

(57) ABSTRACT

The invention provides a novel herbal preparation comprises of Glycine max active fraction containing 7S globulin protein extract, Curcuma longa and Zingiber officinale Linn. rhizome extract used in treatment of diabetes and diabetic related diseases.

### FIELD OF THE INVENTION

The invention provides a novel herbal preparation comprises of Glycine max active fraction containing 7S globulin protein extract, Curcuma longa and Zingiber officinale Linn. Rhizome extract used in treatment of diabetes.

### BACKGROUND AND PRIOR ART

Diabetic mellitus is a chronic condition that is diagnosed by a blood test and requires life long management (American Diabetic association, 2002). The more patients understand about the disease the better they are enabled to make good decisions on its management. Dietary therapy and exercise are critical both in preventing and managing diabetic mellitus and the results of the diabetic prevention program research group indicate that changes in life style reduced the incidence of diabetic by 58% (Knowler et al., 2002). In type 1 diabetic mellitus, where there is an absolute deficiency of insulin replacement forms a major component of treatment. In type 2 diabetic mellitus, insulin release from the pancreas is altered and may also be absolutely deficient in amount, and therefore its replacement also plays a part in management, especially when diabetic mellitus has been present for a long time. As the number of people with diabetes multiply world wide, the disease takes an ever-increasing proportion of national and international health care budgets. It is projected to become one of the world's main disablers within the next 25 years. It is very popularly known in medical history as “silent killer”. Regions with greatest potential are Asia and Africa, where diabetes mellitus rates could rise to two to three-folds than the present rates. Apart from currently available therapeutic options, many herbal medicines have been recommended for the treatment of diabetes and diabetic related diseases (Sabu and Kuttan, 2002; Zhang and Tan 2000; Chitra et.al., 1998; Padma et.al., 2000; Osadebe et.al., 2004; Obatomei et.al., 1994; Oojewole and Adewunni 2004). Plant medicines are used throughout the world for a range of diabetic presentations.

The development of scientifically validated models of alloxan induced-diabetic is vital to the analysis of the functional consequences of pancreatic damage and to testing the recovery efficacy of potentially therapeutic drugs. The role of medicinal plants in increasing the secretion of insulin and acting as an anti-diabetic formulation in the form of tablet is still much underestimated. The retrieval hypothesis postulates the alloxan agents disrupt the pancreas function as the effect of alloxan agents diminish over time by the treatment of our present herbal formulation investigation resulted in the reappearance of normal functioning of pancreas. Drugs like glibenclamide, penformin-containing substances which has a stimulant activity on diabetic. Accordingly, studies shown that the herbal formulation having the property of improving the functioning of pancreas by alloxan induced diabetic and used in treatment of diabetic as a tablet and acting as a strong anti-diabetic formulation.

### OBJECT OF THE INVENTION

The main object of the present invention is to provide a novel herbal combination in dosage form used as a anti-diabetic tablet, injection and formulation.

Another objective of the present invention is to prepare herbal dosage form that improves in the treatment of diabetic mellitus.

Yet another object of the present invention is to prepare herbal dosage from in the form of tablet for easy consumption.

### SUMMARY OF THE INVENTION

Accordingly the present invention provides an herbal formulation useful in the treatment of herbal dosage form from the soya bean milk used as a anti-diabetic. The herbal formulation comprising of Glycine max active fraction containing 7 S globulin fraction extract, Zingiber officinlis (rhizome) and curcumin (a phenolic antioxidant). Glycine max (Soya bean) protein varies from light cream to white in colour. It is used as nourishing food. It was shown that it produce a significant improvement in general ability and behavioural pattern.

Glycine max Family:

Leguminosae

Botanical description: An annual with erect or climbing stem reach a height of one half to six feet, densely clothed with leaves trifoliate, ovate-lanceolate, inconspicuous, borne on auxiliary racemes, white or purple to red purple, normally self pollinated; pods 3 cm long in clusters of 3-5, densely hairy containing 3-4 seeds elliptical with hilum, compressed, yellow, choclate or black. Soyabean is a native of south-eastern Asia is considered, on the basis of genetical have originated from slender, prostste plan. Soyabean is an important legume crop in Far East.

Medicinal uses: Soyabean ranks high among the leguminous crops of the world. It is grown mainly as a food crop of the world. It is grown mainly as a food crop in China, Japan and other country of East Asia. The seeds are consumed green, dry or sprouted, whole or split. The green seed are used as vegetable; roasted and salted seeds are used in cakes and candies. The seeds are ground in to flour and used for bakery products. The fatty oil extracted from the seed is used for the industrial purposes. The soya been used as a whole been, or processed as a soya milk, tofu, tempesh, soya sauce. The increasing popularly of soya food in mainly used in prevention of chronic diseases continues to be a top priority for scientist around the world. Even the FDA has conformed that the food containing the soya protein may reduce the risk of coronary heart diseases. There has been increasing interest of soya been as an antioxidant effect (Wealth of India, 1992).

Phytochemistry: Soya been seeds contain protein 29.6-50.3, fat 13.5-24.2, fiber 2.84-2.67, carbohydrate 14.07-23.88. The decorticated bean contains about 12% polysaccharides. It contains higher % of proteins than many other foodstuffs. Chief protein is a globulin, glycinine that accounts for 80-90% of the total protein of the seed. Besides the true proteins, it contains the following nitrogenous substances like adenine; arginine, glycine etc and total non-protein nitrogen varies from 2.8-6.8% of the total nitrogen. The average mineral composition of mature soya been is as Fe, K, Zn, I etc. it is a good source of beta amylase. It contains variety of pigments, isoflavones, glycosides etc.

Pharmacology: In human metabolism experiments soya been proteins comparable to other pulse protein, biological values digestibility coefficient. Soya been is valued in special diet and as an aid in reliving acidosis. The increasing popularly of soya food in mainly used in prevention of chronic diseases continues to be a top priority for scientist around the world. Even the FDA has conformed that the food containing the soya protein may reduce the risk of coronary heart diseases. Their has been increasing interest of soya been as an antioxidant effect and the particular the isoflavones. (Wealth of India, 1992)

CURCUMA LONGA Family:

Zingiberaceae

Botanical description: The genus Curcuma comprising about fifty species, distributed in tropical and subtropical regions of Asia, belongs to the tribe Hedychieae and consists of a rather homogenous group of rhizomatous perennials. Govindarajan (1980 Food Science and Nutrition, 14: 119-301 and 1982 Food Science and Nutrition, 17:1-258) published critical reviews on turmeric C. longa. The taxonomic status of Curcuma heyneana was discussed by Firman et al (1988 Phytochem. 27: 3887-3891) based on essential oil analysis. Tomlinson's (1969) work based on the anatomical evidence, which has much relevance in the classification of the order Zingiberales.

Medicinal uses: Ethnobotanical details of some of the species of Curcuma has been reviewed and it was found that Curcuma is useful in the treatment of liver disorders and has a promising kind of broad spectrum hepatoprotective agent which is used in Indonesia (Lin et al., American J. Chin. Med., 1995 23:243-254). Curcuma longa was used predominantly for endoparasites, internal and external injuries and pregnancy related conditions in ethnic community of Trinidad and Tobago. Curcuma longa is used as dietary intake in Nepal (Eigner and Scholz, J Ethnopharmacol 1999, 67(1): 1-6).

Phytochemistry: Essential oils are complex mixtures of odorous and steam-volatile compounds that are deposited in the subcuticular space of glandular hair, cell organells, idioblasts, excretory cavities and canals or exceptionally in heartwoods. In other words, they are very complex, aromatic, volatile mixture containing many different compounds. The constituents of essential oils belong to numerous classes of chemical substances, such as hydrocarbons, alcohols, aldehydes, ketones, acids, esters, oxides and ether (Thappa et al, J. Essent. Oil Res., 1982, 11: 97-103). Essential oils largely comprises of terpenoid compounds, which constitute two or more isoprene units. Based on this, terpenoids are mainly classified into four groups viz. monoterpenes (with 2 isoprene units i.e. 10 carbon atoms) sesquiterpenes (with 15 carbon atoms), diterpenes (with 4 isoprine units i.e. 20 carbon atoms) and polyterpenes (with 5 or more isoprene units). These terpenoid compounds provide aroma and pungency to plants. The essential oil forms the basic raw materials for perfume and flavour making industries. They are also used in the cosmetics and pharmaceutical industries. Many natural essential oils are used in aromatherapy to cure and prevent illness due to their therapeutic properties and also because of their fragrance which can influence human thoughts and emotions. Many of the essential oils are reported to have antimicrobial, insect repellent and insecticidal properties.

Pharmacological use: The genus Curcuma exhibits diverse pharmacological activities against cancer and tumorgensis. Anto et al, Mutation Res., 1996, 370:127-131, has reported the anticancer and antitumour properties of Curcuma longa. It was demonstrated that the inhibitory effect of curcumin on DNA and RNA synthesis in cultured HeLa cells. Dietary curcumin may inhibit azoxymethanol (40 M) induced colonic neoplasia in mice (Huang et al., Cancer Lett 1992, 64(2):117-21). The antimicrobial properties are well known and the result reported by many researchers pointed out the antibiotic activities of Curcuma. Banerjee and Nigam (J. Res. Ind. Med. Yoga Homoeo., 1978, 13: 63-70) reported the antibacterial and antifungal activity of various species of Curcuma. Molluscicidal property of C. longa was reported. The insecticidal property of different species of Curcuma. Curcumin showed anti-inflammatory effect in acute, subacute and chronic models of inflammation in mice and rat models. The oral ED₅₀ in mice, against carrageenin-induced acute oedema was 100.2 mg/kg compared to 78 mg/kg of cortisone. Clinically curcumin did not produce any side effect up to 1600 mg/kg/day for 4 weeks in phase-I trials in male volunteers. Phase-II clinical trials have been conducted in patients with rheumatoid arthritis and osteoarthritis. Curcumin inhibited rat liver microsomal delta 5 and delta 6 denaturizes (Shimizu et al., Lipids 1992, 27(7):509-12). Curcuma contains an active principle(s) other than curcuminoid, which can modify the metabolism of lipid and lipoproteins. Several reports suggest that curcumin as well as turmeric increase bile flow. Essential oils of turmeric have also been found to increase the bile flow. However, some investigators have found it to be ulcerogenic (Prasad et al. J. Physiol. Pharmocol, 1976, 20, 92). The gastric secretion was found to be reduced after 3 h in conscious rabbits by aqueous and methanolic extracts of turmeric (Sakai et al. Chem. Pharm. Bull. 1989, 37, 215). Curcumin and turmeric have been shown to protect liver against a variety of toxicants in vitro as well as in vivo. They include carbon tetrachloride, aflatoxin B-1, paracetamol iron, and cyclophosphamide in mouse, rat and duckling. Evidence for the hypocholesterolemic and hypolipidemic activities of curcumin has been provided when it was fed with diet to rats for 7 weeks at the concentration of 0.15% (Rao et al. 1970 J. Nutri. 100, 1307). Ethanolic extract of C. longa has been shown to have hypoglycemic activity in normal as well as alloxan-induced diabeties in rats. They have also isolated a lipopolysaccharide from the root of Curcumin, which is similar to bacterial lipopolysaccharides and is immunostimulant (Inagawa et al. Chem Pharm Bull 1992, 40, 1994). The wound healing property of turmeric was investigated long back and its local application was found to be effective (Gujral et al., J. Ind. Med. Association 22, 273 1958). A sum of approximately 26 compounds has been isolated from different Curcuma sp. having high antioxidant activity. Curcumin did not produce any toxicity either on single administration or on repeated oral administration over a period of 6 months in rat and monkey at doses up to 800 and 1800-mg/kg day, respectively. Curcumin administered orally to patients suffering from chronic antieri or ureitis (CAU) at a dose of 375 mg three times a day for 12 weeks and all the patients who received curcumin alone improved (Lal et al., Phytother Res 13(4): 318-22, 1999).

Zingiber officinale Linn. Family: Zingiberaceae

Botanical description: An herbaceous rhizomatous perennial, reaching up to 90 cm in height under cultivation. Rhizomes are aromatic, thick lobed, pale yellowish, bearing simple alternate distiches narrow oblong lanceolate leaves. The herb develops several lateral shoots in clumps, which begin to dry when the plant matures. Leaves are long and 2-3 cm broad with sheathing bases, the blade gradually tapering to a point. Inflorescence solitary, lateral, radical, pendiculate oblong-cylindrical spikes. Flowers are rare, rather small, calyx superior, gamosepalous, three toothed; open splitting on one side, and corolla of three-sub equal oblong to lanceolate connate greenish segments. (The Wealth of India, NISCOM, D-23, C.S.I.R., New Delhi 1996).

Medicinal use: Ginger is carminative, pungent, stimulant, used widely for indigestion. It is chiefly used to cure diseases due to morbidity of kapha and vata. Ginger with limejuice and rock salt increases appetite and stimulates the secretion of gastric juices. It is said to be used for chronic bronchitis, cold, chest congestion, and cough, difficulty in breathing, dropsy, sore throat, throat ache, stomachache, vomiting and rheumatism. Zinger forms an important constituent of many pharmacopoeal ayurvedic formulations. (Misra B, Bhavaprakasha Nighantu, 5^{th} ed., 1969, p. 14; Sharma P, Dravyaguna vigyan, part 2, Chauukhamba Publications 1993, p. 331; Indian medicinal plant, a compendium of 500 sps., Part 5, orient Longman Publication, 1997, p. 431; Nadkarni, Indian Materia Medica, vol. 1, 1993, p. 1308; Yogaratnakra, Chauukhamba Publications 1993, p. 320-330; Bhavaprakasha with Vaidyotina commentary by Misra B S; Chikitsa Prakarana Madhyana Khadana—Chauukhamba Publications 1980, p. 683-701).

Phytochemistry: Ginger has been reported to contain usually 1-3% of volatile oil, pungent principles, viz. gingerols and shogaols and about 6-8 lipids and others. Ginger oil contains zingiberene and bisaboline as major constituents along with other sesqui and mono terpines. Ginger oleoresin contains mainly the pungent principles gingerols and shogaols as well as zingeberine. Shogaols have recently been found twice as pungent as gingerols. (Kiuchi F, et. al., Chem. Pharm. Bull, 1982, 30, 754; waigner H, et al., Plant drug analysis, springer, 1996, 300; Akhila A and Tewari CROMAP, 1984, 6(3), 143-146).

Pharmacology: It is used for common cold, due to pathogenic wind cold, characterized by severe intolerance to cold, slight fever, headache, general ache, congestion and running nose. Antihistaminic activity has been studied in ginger. Zingiber officinale was indicated in allergic conditions in traditional text. However they were following crude methods. Toyoda J, Chem. Abst., 1969, 71, 33425; Yogaratnakra, Chauukhamba Publications 1993, p. 320-330; Bhavaprakasha with Vaidyotina commentary by Misra B. S; Chikitsa Prakarana Madhyana Khanda—Chauukhamba Publications 1980, p. 683-701).

### BRIEF DESCRIPTION OF THE TABLES

Table I: Effect of formulation (Fl) Glycine max (soya bean) active fraction containing 7S globulin fraction on alloxan induced diabetic rats.

Table II: Effect of formulation (F2) containing Glycine max (soya bean) active fraction containing 7S globulin fraction with Curcuma longa extract on alloxan induced diabetic rats.

Table III: Effect of formulation (F3) containing Glycine max (soya bean) active fraction containing 7S globulin fraction, Curcuma longa with Zingiber officinlis on alloxan induced diabetic rats.

Table IV: Effect of formulation (F4) with out Glycine max (soya bean) active fraction containing 7S globulin fraction on alloxan induced diabetic rats.

Table V: Effect of formulation (F3) containing Glycine max (soya bean) active fraction containing 7S globulin fraction, Curcuma longa, Zingiber officinale on relative mean±SEM organ weights of rats (n=6).

Table VI: Effect of formulation (F3) containing Glycine max (soya bean) active fraction containing 7S globulin fraction, Curcuma longa and Zingiber officinale on Glucose tolerance test in rats.

### DETAILED DESCRIPTION OF THE PRESENT INVENTION

Accordingly, the present invention provides a novel of the invention of an herbal formulation(s) obtained from decoction of two most potent plants having the property of anti-diabetic and used in treatment of diabetes mellitus as a tablet, injection and as a liquid formulation(s).

In another embodiment of the present invention the novelty of the invention is a said herbal formulation having the property of anti-diabetic and used in treatment of diabetic mellitus as a tablet and as a formulation.

Further, in another embodiment of the invention, an herbal anti-diabetic synergistic formulation(s) containing extracts of plant in pharmacologically effective form.

In another embodiment of the invention, herbal formulation(s) the plants selected from the genus Glycine max (2-5 wt %).

In another embodiment of the invention, the 7S globulin fraction of Glycine max seed.

In another embodiment of the invention, the plants selected from the genus Curcuma longa (1-3 wt %).

In another embodiment of the invention, the plants selected from the genus Zingiber officinale (1-3.5 wt %).

In another embodiment of the invention, the composition as a soft/hard gelatin capsule of oral dosage forms.

In another embodiment of the invention, the extract of Glycine max is of active fraction containing 7 S globulin fraction extracts, Zingiber officinlis and Curcuma longa is of rhizome extracts.

In another embodiment of the invention, the formulation having the property of improving the anti-diabetic property and used in treatment of type II diabetes and type I diabetes.

In another embodiment of the invention, the formulation is used as anti-oxidant, cooling, oleaginous, astringent, nerves relaxant properties and anti-diabetic property.

In another embodiment of the invention, the formulation is use to treat diabetic related diseases, blood purifier, anti-periodic and externally applied sprain and wound.

In another embodiment of the invention, the said formulation is used as a tonic.

In another embodiment of the invention, the said formulation is use as an anti-parasitic for many skin affections.

In another embodiment of the invention, the said formulation is use as antacid, carminative.

In another embodiment of the invention, the said formulation is use as cholerectic action.

In another embodiment of the invention, the said formulation is use as anti-arthritis.

In another embodiment of the invention, the said formulation further comprises the specific gravity 0.992-1.505.

In another embodiment of the invention, the formulation further comprises the refractive index of 1.5463-1.6914.

In another embodiment of the invention, the formulation used in cakes and candies.

In another embodiment of the invention, the said formulation further comprises proteins, 80.6-90.7%.

In another embodiment of the invention, the formulation further comprises Amino acids 1.0-19.0%

In another embodiment of the invention, the said formulation further comprises the fibre 2.845-6.27%.

In another embodiment of the invention, the formulation further comprises the phosphorus, 0.69%

In another embodiment of the invention, the formulation further comprises fat, 13.5-24.2%.

In another embodiment of the invention, the formulation further comprises carbohydrates, 14.3-23.5%.

In another embodiment of the invention, the suspension at a dose of 50 to 200 mg/kg did not show any abnormality of general condition in treatment as anti-diabetic activity.

In another embodiment of the invention, the suspension at a dose of 50 to 200 mg/kg on anti-diabetic showed significant and dose dependent activity.

In another embodiment of the invention, the suspension at a dose of 50 to 200 mg/kg showed significant and dose dependent antioxidant activity.

In another embodiment of the invention, the liquid dosage form of Glycine max at a dose of 50-200 mg/kg showed an 11.91-26.65% protection in diabetes.

In another embodiment of the invention, the liquid dosage form of Glycine max at a dose of 50-200 mg/kg shown a 28.77% glucose tolerance.

In another embodiment of the invention, the suspension form of Glycine max at a dose of 50-200 mg/kg shown a 19.5-45.53% protection in diabetes dose dependently with the combination of Glycine max with Curcuma longa.

In another embodiment of the invention, the suspension form of Glycine max at a dose of 50-200 mg/kg shown a 29.8-55.0% protection in diabetes dose dependently with the combination of Glycine max, Curcuma longa with Zingiber officinalis.

The invention thus meets the need for a new process in which the optimal proportions of vitamins, amino acids, long chain fatty acids and active therapeutic marker compounds are retained in the product and underlies the efficacy of the compound as anti diabetic.

Further in another embodiment of present invention the Glycine max (soya bean) seed fraction containing 7S globulin-like protein (Komatsu and Hiranc, 1991) according to the invention can be incorporated into a variety of food products, including, without limitation, butter, cakes, candies, ice cream and mayonnaise-chocolate products, preparation of jaggery, water based brinks such as wines and mineral waters. The inventive oil is also suitable for encapsulation in gelatine shells to form soft gels/capsules. Regardless of the particular form in which the inventive oil is prepared, the daily dosage of the fraction to experimental animals fall within the ranges set forth above. Depending on the concentration of the inventive protein fraction in the above form, the total amount of the food product per serving or encapsulated will also vary the desired therapeutic activity.

### EXAMPLES

The invention is further illustrated by the following non-limiting examples.

Formulation 1 (F1)

| | | |
|---|---|---|
| Glycine max (Soya bean) | 3 | wt. % |
| Lactose | 66.7 | g |
| Starch | 10 | g |

| | |
|---|---|
| Water | q.s. to make 100 ml |

Dry mature seed of Glycine max (Soya bean) are washed in purified distilled water of pyrogen free, and were immersed in hot water (30-60° C.) for 2-3 hr. the seeds released 7 S globulin were used along with the extract that obtained after squeezed in a silicon cloth to get a white exudates.

Mix the plant constituents and filter the solution and add specified quantity of starch and heat until the starch dissolves and then cool and make up the volume with required amount of water to make 100 ml.

The formulation is useful to a anti-diabetic. Accordingly, the investigation deals with the oral dosage form have been described in detail giving the formula of the ingredients along with the method and mode of usage of the standardized formulation.

Formulation 2 (F2)

| | | |
|---|---|---|
| Glycine max (Soya bean) | 3 | wt. % |
| Curcuma longa | 1.5 | wt. % |

| | |
|---|---|
| Lactose | 2.5% |
| Starch | 0.5% |
| Water | q.s. to make 100 ml |

Dry mature seed of Glycine max (Soya bean) are washed in purified distilled water of pyrogen free, and were immersed in hot water (30-60° C.) for 2-3 hr. the seeds released 7 S globulin were used along with the extract that obtained after squeezed in a silicon cloth to get a white exudates. The Curcuma longa were collected and dried in shade. The dried material (1 Kg) is then powdered and extracted with water for 5 days. At the end of this, the solvent is decanted and filtered if necessary to remove the plant debris. The extract is then concentrated under vacuum at less than 50° C. Then the extract is lyophilized to obtain the extract in powder form.

Mix the plant extracts and dissolve them water, filter the solution and add specified quantity of starch and heat the until the starch dissolves and then cool and make up the volume with required amount of water to make 100 ml. The formulation is useful to a anti-diabetic. Accordingly, the investigation deals with the oral dosage form have been described in detail giving the formula of the ingredients along with the method and mode of usage of the standardized formulation.

Formulation 3 (F3)

| Glycine max (Soya bean) | 3 | wt. % |
|---|---|---|
| Curcuma longa | 1.5% | |
| Zingiber officinale | 1.5% | |
| Lactose | 45.7 | g |
| Starch | 10 | g |

| | |
|---|---|
| Water | q.s. to make 100 ml |

Dry mature seed of Glycine max (Soya bean) are washed in purified distilled water of pyrogen free, and were immersed in hot water (30-60° C.) for 2-3 hr. the seeds released 7 S globulin were used along with the extract that obtained after squeezed in a silicon cloth to get a white exudates. The Curcuma longa and Zingiber officinale were collected and dried in shade. The dried material (1 Kg) is then powdered and extracted with water for 5 days. At the end of this, the solvent is decanted and filtered if necessary to remove the plant debris. The extract is then concentrated under vacuum at less than 50° C. Then the extract is lyophilized to obtain the extract in powder form.

Mix the plant extracts and dissolve them water, filter the solution and add specified quantity of starch and heat the until the starch dissolves and then cool and make up the volume with required amount of water to make 100 ml.

The formulation is useful to a anti-diabetic. Accordingly, the investigation deals with the oral dosage form have been described in detail giving the formula of the ingredients along with the method and mode of usage of the standardized formulation.

Formulation 4 (F4)

| | |
|---|---|
| Curcuma longa | 1.5% |
| Zingiber officinale | 2.5% |
| Lactose | 20 g |
| Starch | 10 g |
| Water | q.s. to make 100 ml |

The Curcuma longa and Zingiber officinale were collected and dried in shade. The dried material (1 Kg) is then powdered and extracted with water for 5 days. At the end of this, the solvent is decanted and filtered if necessary to remove the plant debris. The extract is then concentrated under vacuum at less than 50° C. Then the extract is lyophilized to obtain the extract in powder form. Mix the plant extracts and dissolve them water, filter the solution and add specified quantity of starch and heat the until the starch dissolves and then cool and make up the volume with required amount of water to make 100 ml. The formulation is useful to a anti-diabetic. Accordingly, the investigation deals with the oral dosage form have been described in detail giving the formula of the ingredients along with the method and mode of usage of the standardized formulation.

Alloxan-Induced Hyperglycemia

Hyperglycemia was induced by a single intraperitoneal injection of 120 mg/kg of alloxan monohydrate in sterile saline. After five days of alloxan injection, the diabetic rats (glucose level >350 mg/dl) were separated and divided into different groups of six animals each. Blood samples were collected from the tail vein just prior to and 1 and 3 h after Glycine max (soya bean) administration. And same procedure repeated for effect of formulation with and with out Glycine max (soya bean) protein on alloxan-induced rats. (Venkatesh et al, 2003)

Effect Formulation on Glucose Tolerance in Rats

Fasted rats were divided into four groups of six rats each. Groups of rats were treated with the formulation and after 30 min of the rats of all groups were orally treated 2 g/kg of glucose. Blood samples were collected from the tail vein just prior to glucose administration and at 30 and 90 min after glucose loading. Serum was separated and blood glucose levels were measured immediately by glucose oxidase method. (Venkatesh et al, 2003)

TABLE I

| | Exper- | Blood glucose (mg/100 ml) | | |
|---|---|---|---|---|
| Group | iment | Basal value | 1 h | 3 h |
| I | Diabetic | 355.08 ± 18.66 | 348.42 ± 20.91 | 342.02 ± 21.16 |
| | control | | | |
| | (distilled | | | |
| | water) | | | |
| II | F1 (50 | 346.08 ± 17.11 | 325.75 ± 18.33 | 309.24 ± 19.22 |
| | mg/kg) | | | |
| III | F1 (100 | 389.54 ± 16.33 | 340.61 ± 17.01 | 305.99 ± 18.22^{a} |
| | mg/kg) | | | |
| IV | F1 (200 | 392.77 ± 13.17 | 348.61 ± 16.58 | 288.11 ± 17.44^{b} |
| | mg/kg) | | | |

Values are mean ± S.D. for six rats;
P ^{a}< 0.01 and ^{b}< 0.001 compared to respective basal value group.
F1- Formulation containing only Glycine max

The results of the present study of table-I shows that there is a significant decrease in blood glucose level at 100 mg/kg and 200 mg/kg of containing only Glycine max (Soya bean) active fraction containing 7S globulin fraction in alloxan induced diabetes at 3 h and percentage protection ranged 11.91-26.65 percentage in controlling the diabetes.

TABLE II

| | Exper- | Blood glucose (mg/100 ml) | | |
|---|---|---|---|---|
| Group | iment | Basal value | 1 h | 3 h |
| I | Diabetic | 345.08 ± 15.66 | 350.42 ± 22.91 | 354.02 ± 18.16 |
| | control | | | |
| | (distilled | | | |
| | water) | | | |
| II | F2 (50 | 366.08 ± 15.11 | 334.75 ± 17.33 | 306.24 ± 18.22^{a} |
| | mg/kg) | | | |
| III | F2 (100 | 384.08 ± 19.11 | 337.58 ± 17.01 | 275.24 ± 16.89^{b} |
| | mg/kg) | | | |
| IV | F2 (200 | 372.54 ± 19.08 | 315.61 ± 16.54 | 255.99 ± 14.65^{c} |
| | mg/kg) | | | |

Values are mean ± S.D. for six rats;
P ^{a}< 0.05, ^{b}< 0.01 and ^{c} < 0.001 compared to respective basal value group.
F2- Formulation containing Glycine max (soya bean) active fraction containing 7S globulin fraction with Curcuma longa extract.

The results of the present study of table-II shows that there is a significant decrease in blood glucose level at a dose range 50-100 mg/kg showed significant results and the percentage protection ranged 19.5-45.53 percent in lowering the increased level of blood glucose level.

TABLE III

| | Exper- | Blood glucose (mg/100 ml) | | |
|---|---|---|---|---|
| Group | iment | Basal value | 1 h | 3 h |
| I | Diabetic | 352.20 ± 21.01 | 355.60 ± 23.10 | 353.80 ± 23.80 |
| | control | | | |
| | (dis- | | | |
| | tilled | | | |
| | water) | | | |
| II | F3 (50 | 358.30 ± 18.95 | 342.20 ± 17.33 | 251.60 ± 18.22^{a} |
| | mg/kg) | | | |
| III | F3 (100 | 360.10 ± 19.21 | 330.60 ± 18.91 | 210.51 ± 15.55^{b} |
| | mg/kg) | | | |
| IV | F3 (200 | 365.20 ± 17.99 | 215.61 ± 15.64^{b} | 166.99 ± 13.65^{c} |
| | mg/kg) | | | |

Values are mean ± S.D. for six rats;
P ^{a}< 0.05, ^{b}< 0.01 and ^{c}< 0.001 compared to respective basal value group.
F3- Formulation containing Glycine max (soya bean) active fraction containing 7S globulin fraction, Curcuma longa with Zingiber officinale

The results of the present study of table-III shows a significant decrease in blood glucose level at 1 h at a dose 200 mg/kg and the percentage protection in controlling the increased level of blood glucose were significant at dose range 50-200 mg/kg at 3 h and percentage protection ranged 29.8-55.0 percent.

TABLE IV

| | Exper- | Blood glucose (mg/100 ml) | | |
|---|---|---|---|---|
| Group | iment | Basal value | 1 h | 3 h |
| I | Diabetic | 365.08 ± 19.01 | 358.42 ± 18.35 | 368.12 ± 20.37 |
| | control | | | |
| | (distilled | | | |
| | water) | | | |
| II | F4 (50 | 372.77 ± 15.68 | 368.61 ± 16.58 | 348.11 ± 19.54 |
| | mg/kg) | | | |
| III | F4 (100 | 358.08 ± 14.35 | 325.75 ± 16.24 | 316.24 ± 17.64 |
| | mg/kg) | | | |
| IV | F4 (200 | 365.24 ± 18.02 | 348.15 ± 16.29 | 304.29 ± 14.61^{a} |
| | mg/kg) | | | |

Values are mean ± S.D. for six rats;
P ^{a}< 0.01 compared to respective basal value group.

F4—Formulation contains Curcuma longa and Zingiber officinale without Soya bean active fraction containing 7S globulin fraction.

The results of the present study of table-IV showed the probable significant effect (P<0.05) at 3 h at a dose range of 200 mg/kg. But the results of table III of formulation F3 is highly significant even when we compared with the percentage protection of that shown 29.8-55.0 percent.

TABLE V

| Type of | Treat- | Body | | | |
|---|---|---|---|---|---|
| treat- | ment | weight | | | |
| ment | group | (g) | Kidney (g) | Liver (g) | Spleen (g) |
| 6 days | Control | 172.0 ± | 0.83 ± 0.03 | 5.73 ± 0.45 | 0.59 ± 0.06 |
| oral | | 10.3 | | | |
| treat- | F3 (50) | 164.3 ± | 0.91 ± 0.03 | 5.56 ± 0.59 | 0.71 ± 0.04 |
| ment | | 9.6 | | | |
| | F3 (100) | 162.9 ± | 0.87 ± 0.07 | 5.76 ± 0.55 | 0.75 ± 0.01 |
| | | 13.3 | | | |
| | F3 (200) | 167.5 ± | 0.85 ± 0.09 | 5.71 ± 0.61 | 0.59 ± 0.05 |
| | | 10.0 | | | |

F3- Formulation containing Glycine max (soya bean) active fraction containing 7S globulin fraction, Curcuma longa, Zingiber officinale.
Note:
No mortality/gross abnormality were observed in the animals during the treatment of formulation F3.

TABLE VI

| | | Blood glucose (mg/100 ml) | | |
|---|---|---|---|---|
| Group | Experiment | Fasting | 30 min | 90 min |
| I | Glucose (2 g) | 87.08 ± 1.72 | 165.43 ± 4.98 | 134.65 ± 4.33 |
| II | F3 (50 mg/kg) | 82.06 ± 1.45 | 145.13 ± 3.75 | 138.09 ± 2.54 |
| III | F3 (100 mg/kg) | 86.71 ± 1.63 | 152.54 ± 3.56 | 125.75 ± 2.76^{a} |
| IV | F3 (200 mg/kg) | 79.82 ± 2.12 | 143.51 ± 3.58 | 102.22 ± 3.65^{b} |

Values are mean ± S.D. for six rats;
P ^{a}< 0.01 and ^{b}< 0.001 compared to respective basal value group.

The results showed a significant effect with the formulation F3 containing Glycine max (Soya bean active fraction containing 7S globulin fraction), Curcuma longa and Zingiber officinale at 90 min and the levels were revealed near to the normal values in the scientifically validated model of glucose tolerance test in rats.

### REFERENCES

Agarwal et al., Indian Journal of Experimental Biology, 38, pp994-998, 2000.

Asres K, Indian Drugs, 30, pp 189-190,1993.

Barik et al, Indian Journal of Chemistry, 20B, pp 938-939, 1981.

Bean et al, Journal of Natural Product, 48, pp 500, 1985.

Chitra P, Sajithlal G B, Chandrakasan G. Influence of Aloevera on the healing of dermal wounds in diabetic rats. J. Ethanopharmacol. 1998; 59: 195-201.

Chopra R N et al, Glossary of Indian Medicinal Plants, 1956.

Chopra R N et al, Indian journal of medical research, 29, pp 769, 1941.

Chottopadhyay R R, Indian Journal Of Experimental Biology, 31,pp 893, 1993.

Holmann et al, Textbook of Diabetics, pp 467-469, 1991,

Joy K L, Kuttan R, Journal of Ethnopharmacology, 67(2), pp 143, 1999.

Kameshwara Rao et al, manphar vaidhya partika, 1, pp 33, 1997.

Kamiya et al, Phytochemistry, 57, pp 297-301, 2001.

Kirthikar K R and Basu B D, Indian Medicinal plants, 1, 1981.

Obatomi D K, Bikomo E O, Temple V J. Anti diabetic properties of the African mistletoe in STZ induced diabetic rats. .J. Ethanopharmacol. 1994; 43(1): 13-17.

Osadebe P O, Okide G B, Akabogu I C. Study on anti diabetic activities of crude methanolic extracts of Lorathus micranthus (Linn) sourced from five different host trees. .J. of Ethanopharmacol. 2004; 95(2-3): 133-138.

Padma P, Khosa R L, Chansouria J P N. Lipid peroxidation during healing of cutaneous wounds in albino rats. Ind. J. of Natural Products. 2000; 18(2): 16-19.

Pari L, Journal of Ethnopharmacology, 68(1), pp 321, 1999.

Romesh P, Yuvarajan C R, Journal of Natural Product, 58, pp 1242-1243, 1995.

Sabu M C, Kuttan R. Anti diabetic activity of medicinal plants and its relationship with their anti oxidant property. J. Ethanopharmacol. 2002; 81(2): 155-160.

Saraswati Bai N, Bull. Cent. Res. Inst, 3, pp 89-107, 1954.

Satake et al, Chem. Pharma Bull, 47, pp 1444-1447, 1999.

Setsuko Komatsu and Hisashi Hiranc, Federation of European Biochemical sciences 294(3) 210-212, 1991.

Singh et al, Indian Journal of Pharmaceutical Sciences, 46, pp 148-149, 1984.

Subramanyan, Current Science, 14, pp 204, 1945.

Thorpe, Chemurg Dig, 10 (2), pp 11, 1951.

Trease and Evan, Textbook of Pharmacognosy, 1983.

Trinder P, Ann Clinical Biochem., 6, pp 24-27, 1969.

U.S. Pat. No. 6,592,896 July, 2003 Rosen bloom

U.S. Pat. No. 6,596,313 July, 2003 Rosen bloom

U.S. Pat. No. 6,632,459 October, 2003 Graus, et. al.

U.S. Pat. No. 6,649,185 November, 2003 Solanki

Valiathan M S, current science, 75, pp 1120-1122, 1998.

Venkatesh et al, Fitoterapia, 74, pp. 274-279, 2003.

Venkatesh et al, Pharmaceutical Biology, 41, 2003.

Zhang X F, Tan B. K. Anti hyperglycaemic and anti oxidant properties of Androgaphis paniculata in normal and diabetic rats. Clin. Exp. Pharmacol. Physiol. 2000; 27 (5-6): 358-363.

We claim:

1. A formulation comprising an extract of Glycine max in the range of 2-5 wt % along with extract(s) of Curcuma longa in the range of 1-3 wt % and/or Zingiber officinale in the range of 1-3.5 wt % optionally along with pharmaceutically acceptable carrier(s), said formulation is used in treatment of diabetes and diabetes related diseases.

2. A formulation as claimed in claim 1, wherein the Glycine max extract is seed extract.

3. A formulation as claimed in claim 1, wherein the Glycine extract contains 7S globulin.

4. A formulation as claimed in claim 1, wherein the Zingiber officinalis extract is rhizome extracts.

5. A formulation as claimed in claim 1, wherein said formulation is administered to a subject as soft/hard gelatin capsule, injection or oral dosage forms.

6. A formulation as claimed in claim 1, wherein the formulation is used in cakes and candies.

7. A formulation as claimed in claim 5, wherein the subject is mammal and preferably human.

8. A formulation as claimed in claim 1, wherein the said formulation having the property of improving the anti-diabetic property and used in treatment of type II diabetes and type I diabetes.

9. A formulation as claimed in claim 1, wherein the formulation is used as a free radical scavenger, anti-oxidant and anti-diabetic.

10. A formulation as claimed in claim 1, wherein the formulation is used to treat diabetic related diseases, as inflammation and wound.

11. A formulation as claimed in claim 1, wherein the formulation is used as anti-parasitic for many skin infections.

12. A formulation as claimed in claim 1, wherein the said formulation is used as antacid, carminative.

13. A formulation as claimed in claim 1, wherein the said formulation is used as cholerectic action.

14. A formulation as claimed in claim 1, wherein the said formulation is used as anti-arthritis.

15. A formulation as claimed in claim 1, wherein the said formulation having the specific gravity of 0.992-1.505.

16. A formulation as claimed in claim 1, wherein the formulation has a refractive index of 1.5463-1.6914.

17. A formulation as claimed in claim 1, wherein the pharmaceutically acceptable carrier is selected from a group comprising protein, carbohydrate, amino acids, phosphorous, fat and fiber.

18. A formulation as claimed in claim 1, wherein the said formulation further comprises proteins in the range of 80.6-90.7 wt %.

19. A formulation as claimed in claim 1, wherein the formulation further comprises amino acids in the range of 1.0-19.0 wt %

20. A formulation as claimed in claim 1, wherein the said formulation further comprises the fibre in the range of 2.845-6.27 wt %.

21. A formulation as claimed in claim 1, wherein the formulation further comprises phosphorus in the range of 0.55-0.75 wt %.

22. A formulation as claimed in claim 1, wherein the formulation further comprises fat in the range of 13.5-24.2 wt %.

23. A formulation as claimed in claim 1, wherein the formulation further comprises carbohydrates in the range of 14.3-23.5 wt %.

24. A use of formulation of claim 1 in the treatment of diabetes and diabetes related diseases.

25. A use as claimed in claim 24, wherein the suspension form of Glycine max with Curcuma longa and Zingiber officinlis at a dose of 50-200 mg/kg reduces the blood glucose level by at least 55.0%.

26. A use as claimed in claim 24, wherein the said formulation at a dose of 50 to 200 mg/kg does not show any abnormality of general condition in mammal preferably human.

27. A use as claimed in claim 24, wherein the said extract of Glycine max at a dose of 50-200 mg/kg reduces blood glucose level by at least 26.65% in mammal preferably human.

28. A use as claimed in claim 24, wherein the extract of Glycine max at a dose of 50-200 mg/kg show a 28.77% glucose tolerance in mammal preferably human.

29. A use as claimed in claim 24, wherein the extract of Glycine max with Curcuma longa at a dose of 50-200 mg/kg reduce blood glucose level by at least 45.53% in mammal preferably human.
